(19) United States
(12) Patent Application Publication
(10) Pub. No.: US 2011/0015496 A1
(43) Pub. Date: Jan. 20, 2011

(54) PORTABLE MEDICAL DEVICE

(76) Inventors: LAWRENCE M. SHERMAN, Westport, CT (US); Richard Alan Stein, Westport, CT (US); Kenneth Hillel Shubin Stein, New York, NY (US); Charles Sean O'Connor, Norwalk, CT (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/502,746

(22) Filed: Jul. 14, 2009

(51) Int. Cl. A61B 5/00; G08B 23/00

(52) U.S. Cl. 600/301; 340/573.1

(57) ABSTRACT

A housing configured to at least partially encapsulate a mobile communication device, a sensor disposed on the housing, the sensor being configured to collect medical information relating to a patient, and an interface coupled to the sensor and configured to communicate the collected medical information to the mobile communication device.

### BACKGROUND

Early and frequent access to medical care can be one method to prevent thousands of deaths each year. Traditionally, medical care has been limited to formal settings such as hospitals and doctors' offices that possess the desired medical equipment. Requiring patients to go to a formal medical facility has many drawbacks, however, such as increased costs and increased time to receive medical attention. In addition, many medical conditions only appear on a sporadic basis making these conditions difficult to diagnose because symptoms often do not manifest themselves while a patient is being examined by a doctor. For example, some heart irregularities occur randomly and are not reproducible in a controllable manner.

In light of the many drawbacks of requiring formal visits to a doctor, there has recently been a trend to bring medical devices directly to consumers, either for self-monitoring and/or in conjunction a formal treatment program. For example, it is quite common for patients to use self-operated blood glucose meters and blood pressure monitors. Other techniques also exist to provide out-of-office monitoring of many conditions such as heart irregularities using event recorders.

### SUMMARY

In general, in an aspect, the invention can provide a medical device including a housing configured to at least partially encapsulate a mobile communication device, a sensor disposed on the housing, the sensor being configured to collect medical information relating to a patient, and an interface coupled to the sensor and configured to communicate the collected medical information to the mobile communication device.

Implementations of the invention may provide one or more of the following features. The sensor includes a plurality of sensors configured to detect at least one of electrical activity in the patient, physiological parameters, and environmental parameters. The plurality of sensors are configured to collect information used to generate an electrocardiogram. The housing is configured as a skin that at least partially encapsulates the mobile communication device without substantially interfering with the operation of the mobile communication device. The housing includes a feedback device configured to provide to the patient feedback relating to the operation of the medical device. The feedback device is configured to provide feedback relating to at least one of a duration to collect the medical information and a quality of medical information collected. The interface is at least one of a wired interface configured to connect with an interface on the mobile communication device and a wireless interface. The medical device further includes a processor configured to control the operation of at least one of the sensor and the interface. The processor is configured to perform digital signal processing on information provided by the sensor. The interface comprises at least one of a radio frequency identification tag, a tone generator, and a vibration generator.

In general, in another aspect, the invention can provide a tangible computer readable medium including instructions that, when executed, cause a mobile communication device to receive medical information relating to a patient from an external device, perform digital signal processing on the medical information, display on a display of the mobile communication device a visual indication that is derived from the medical information, and store the medical information in a memory.

Implementations of the invention may provide one or more of the following features. The instructions are further configured to cause the mobile communication device to display an electrocardiogram relating to the patient on the display. The medical information is received via a local interface of the mobile communication device, wherein the local interface is at least one of a wired and a wireless interface. The instructions are further configured to cause the mobile communication device to determine whether abnormalities exist in the medical information. The instructions are further configured to cause the mobile communication device to control the operation of the external device. The instructions are further configured to cause the mobile communication device to provide feedback to the patient relating to at least one of the duration to collect the medical information and a quality of medical information. The instructions are further configured to cause the mobile communication device to transmit additional information relating to the medical information via at least one of a mobile telephone network and a data network.

In general, in another aspect, the invention can provide a medical device including a medical information collection device including a first local interface, a sensor in communication with the first local interface and configured to collect medical information relating to a patient, wherein the sensor is configured to provide the medical information to the first local interface, a mobile communication device including a second local interface configured to communicate with the first local interface, a first processor in communication with the second local interface and configured to receive the medical information, the first processor being configured to perform digital signal processing on the medical information, a display in communication with the processor and being configured to display an image derived at least in part from the medical information, a network interface in communication with the processor and configured to communicate with at least one of a mobile telephone network and a data network, and a memory in communication with the first processor and being configured to store the medical information, wherein the medical information collection device is configured as a housing that at least partially encapsulates the mobile communication device.

Implementations of the invention may provide one or more of the following features. The sensor comprises a plurality of sensors configured to detect at least one of electrical activity in the patient, physiological parameters, and environmental parameters. The first processor is configured to generate an electrocardiogram using the medical information. The housing is configured as a skin that at least partially encapsulates the mobile communication device without interfering with the operation of the mobile communication device. The medical device further comprises a feedback device configured to provide feedback relating to at least one of a duration to collect the medical information and a quality of medical information collected. The first and second interfaces are configured to connect with each other using at least one of a wired and a wireless interface. The medical information collection device further comprises a second processor configured to control the operation of at least one of the sensor and the first interface. The second processor is configured to perform digital signal processing on information provided by the sensor.

In general, in another aspect, the invention can provide a medical device including a housing configured for use with a mobile communication device, a sensor disposed on the housing, the sensor being configured to collect medical information relating to a patient, and an interface coupled to the sensor and configured to communicate the collected medical information to the mobile communication device.

In general, in another aspect, the invention can provide a mobile communication device including a housing, a display, a sensor disposed on the housing and being configured to collect medical information relating to a patient, a processor in communication with the sensor and the display, and a memory including computer readable instructions that are configured to, when executed by the processor, cause the processor to receive the collected medical information, process the collected medical information, and display an image on the display as a function of the collected medical information.

In general, in another aspect, the invention can provide a medical device for use with a mobile communication device including an adhesive skin configured to adhere to at least a portion of the mobile communication device, a sensor disposed on the adhesive skin, the sensor being configured to collect medical information relating to a patient, and an interface coupled to the sensor and configured to communicate the collected medical information to the mobile communication device.

In general, in another aspect, the invention can provide a medical device for use with a mobile communication device including a base portion configured to attach to the mobile communication device, an arm connected to the base portion, a sensor disposed on the arm, the sensor being configured to collect medical information relating to a patient, and an interface coupled to the sensor and configured to communicate the collected medical information to the mobile communication device.

In general, in another aspect, the invention can provide a device including a housing configured to at least partially encapsulate a mobile communication device, a sensor disposed on the housing, the sensor being configured to collect environmental information, and an interface coupled to the sensor and configured to communicate the collected environmental information to the mobile communication device.

Implementations of the invention may provide the following features. The sensor is at least one of a smoke detector, fire detector, carbon monoxide detector, a thermometer, a moisture detector, and a photocell.

Various aspects of the invention may provide one or more of the following capabilities. Medical information can be collected using a smartphone and an external and/or internal sensor. An ECG can be collected using an application installed on a smartphone and one or more sensors. Medical information can be collected using an ordinary everyday smartphone that most people already own. The time required to diagnose a patient can be decreased. Patients can receive more frequent medical care than prior techniques. Persistent monitoring of the patient can be performed more efficiently than prior techniques. Third parties can be automatically notified of normalities and/or abnormalities detected in the collected medical information. Medical information can be communicated to third parties from the smartphone via a cellular network.

These and other capabilities of the invention, along with the invention itself, will be more fully understood after a review of the following figures, detailed description, and claims.

### BRIEF DESCRIPTION OF THE FIGURES

FIG. 1 is a block diagram of a system configured to collect medical information.

FIG. 2 is a block diagram of a system configured to collect medical information.

FIG. 3 is a diagram of an embodiment of the system shown in FIG. 1.

FIG. 4 is a diagram of an embodiment of the system shown in FIG. 1.

FIG. 5 is a diagram of an embodiment of the system shown in FIG. 1.

FIG. 6 is a diagram of an embodiment of the system shown in FIG. 1.

FIG. 7 is a diagram of an embodiment of the system shown in FIG. 1.

FIG. 8 is a diagram of an embodiment of the system shown in FIG. 1.

FIG. 9 is a diagram of an embodiment of the system shown in FIG. 1.

FIG. 10. is a diagram of an embodiment of the system shown in FIG. 1.

FIG. 11 is a functional block diagram of a process for collecting medical information.

### DETAILED DESCRIPTION

Embodiments of the invention provide techniques for collecting medical information using a portable computing device. A portable computing device can be a smartphone that includes a user application configured to collect medical information relating to a patient. The portable computing device collects the medical information using one or more external sensors connected to the smartphone. For example, a smartphone can generate an electrocardiogram (ECG) of a patient using one or more electrical leads connected to the smartphone. The external leads can be contained in a housing (e.g., skin and/or case) that is configured to hold and connect to the smartphone without interfering with the operation of the smartphone. Other embodiments are within the scope of the invention.

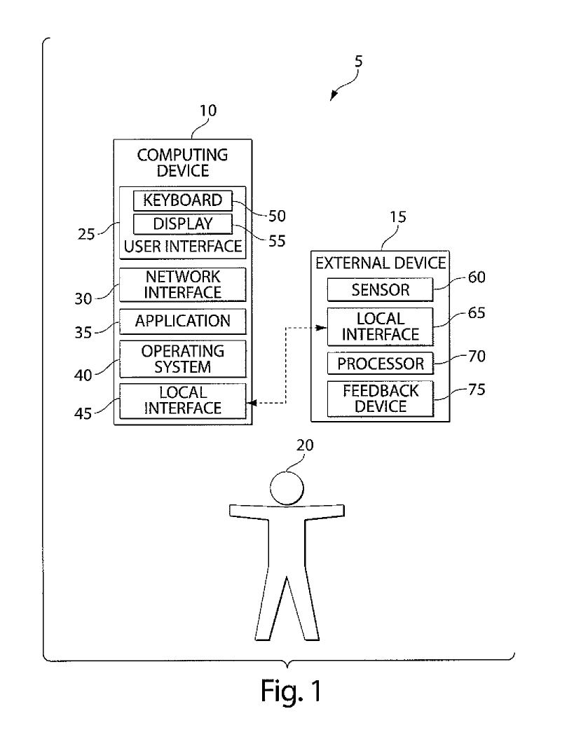

Referring to FIG. 1, a system 5 includes a portable computing device 10 and an external device 15. The system 5 can be configured to monitor one or more medical characteristics of a patient 20. For example, the system 5 can be configured to monitor heart activity (e.g., irregularities), monitor blood glucose levels, monitor blood oxygenation levels, monitor blood pressure, capture ultrasound information, measure body temperature, respiratory rate, basal metabolic rate, active metabolic rate, and/or perform a metabolic stress test. The system 5 can also be configured to provide medical treatment to the patient 20.

The portable computing device 10 is preferably a device that includes a processor, a display, and an interface that can be coupled to and communicate with the external device 15. For example, the computing device 10 can be a device such as a personal computer, a mobile communication device (e.g., an ordinary mobile phone, a smartphone), a portable music player, a portable GPS device, and/or an automobile computer system. Notwithstanding the foregoing, for clarity and not as a limitation, the remainder of this written description will refer to the portable computing device 10 as smartphone 10 and/or IPHONE 110.

Preferably, the smartphone 10 includes a user interface 25, a network interface 30, an application 35, an operating system 40, and a local interface 45. The smartphone 10 can be configured to provide voice and data communication capabilities to the patient 20. Examples of the smartphone 10 include, an APPLE IPHONE, an LG DARE, a BLACKBERRY STORM, and a PALM TREO.

The user interface 25 preferably includes a keyboard 50 and a display 55. The keyboard 50 can be configured to provide the patient 20 with the ability to provide input to the smartphone 10. The keyboard 50 can be, for example, a QWERTY keyboard, and/or a standard telephone keypad. The display 55 is preferably a full color display configured to display information to the patient 20. For example, the display 55 can be configured to display an image of an ECG to the patient 20. The display 55 can also be touch-sensitive (e.g., a touch screen), thereby allowing it also to function as the keyboard 50.

The network interface 30 can be configured to provide a network interface to the smartphone 10. The network interface 30 can be configured to provide data access (e.g., via the Internet and/or an Intranet) and/or voice access (e.g., for telephone calls) to the smartphone 10. The network interface 30 can be configured to connect to a standard telephone network (e.g., GSM, CDMA, and DTMF). The network interface 30 can also be a wireless interface that is configured to connect to the Internet and/or an intranet via a wireless local-area network (e.g., 802.11). The network interface 30 can also be configured to connect to proprietary networks as well.

The application 35 is preferably an application that can be installed on the smartphone 10 and can be configured such that the smartphone 10 can work with the external device 15. The application 35 can be, for example, downloaded via the network interface 30, downloaded via the local interface, and/or installed using other methods. For example, the patient 20 can download the application 35 from a mobile service provider (e.g., AT&T Wireless, T-Mobil, Verizon, Claro, etc.). Alternatively, the application 35 can be provided with the external device 15 (e.g., on an optical disc, a memory card, and/or a SIM). The application 35 is described in more detail below. While FIG. 1 shows application 35 as a separate application from the operating system 40, other configurations are possible. For example, the application 35 can be part of the operating system 40. The application 35 can be certified and/or approved (e.g., by the U.S. Food and Drug Administration) for medical use.

The operating system 40 can be configured to control the overall operation on the smartphone 10. For example, the operating system 40 can be configured to provide a graphical user interface to the patient 20 such that, for example, the patient 20 can make phone calls, send short message service (SMS) messages, and interface with external devices (e.g., the external device 15) via the local interface 45. The operating system can be, for example, SYMBIAN, IPHONE OS, RIM BLACKBERRY, WINDOWS MOBILE, LINUX, GOOGLE ANDROID, and PALM OS. Preferably, the operating system 40 is configured to allow the smartphone 10 to interface with the external device 15 via the local interface 45.

The local interface 45 can be configured to provide data communication with the external device 15. Preferably, the local interface 45 is configured to communicate with one or more external devices located in the general proximity of the smartphone 10. The local interface 45 can be wired (e.g., USB, IEEE 1394, and/or proprietary) and/or wireless (e.g., IEEE 802.11, 802.15, infrared). For example, the local interface 45 can be a 30-pin IPHONE dock connection and/or a personal area network such as IEEE 802.15 (e.g., Bluetooth). The local interface 45 can be configured to send information to and/or receive information from the external device 15.

The external device 15 includes sensor 60 and local interface 65 and is preferably a device that is configured to communicate with the smartphone 10. The external device 15 can be configured to collect information about the patient 20 using the sensor 60 and to provide the collected information to the smartphone 10 via the local interface 45, 65. The external device 15 can be configured to receive power from the smartphone 10 (e.g., via the local interface 45, 65) and/or can receive power from another source (e.g., a separate dedicated power connection and a battery).

The external device 15 can be configured as a housing that is configured to, at least partially encapsulate the smartphone 10. For example, the housing can be a skin and/or case configured to surround, wrap and/or hold the smartphone 10. The external device 15 can be configured as a plastic, rubber, and/or vinyl housing that stretches around the smartphone 10. The external device 15 can also be configured of a rigid material that can slide onto or attach to the smartphone 10 as a “clamshell.” Preferably, the external device 15 is configured such that it does not interfere with the normal operation of the smartphone 10 when installed on the smartphone 10. The external device can also be configured such that, when installed on the smartphone 10, the local interface 65 is positioned to mate with the local interface 45. Alternatively, the external device 15 can also be configured not to interfere with access to the local interface 45. In certain embodiments of the external device 15, the sensor 60 can be disposed directly on the housing itself (e.g., electrical leads on the surface of a skin), and/or can be connected to the housing via, for example, a wire (e.g., the skin can include a connection configured to connect to a blood pressure cuff). The external device 10 can also include a belt clip and/or be configured to slip into a holster that includes a belt clip (e.g., a case within a case).

The external device 15 can also be configured as a stand alone (i.e., not as a housing for the external device 15) medical testing device that connects directly to the smartphone 10. For example, the external device 15 can be a standard set of ECG electrodes and/or a standard finger oxygen sensor that connect to the local interface 45 using an adapter plug. In other embodiments, the external device 15 can be, for example, a wristwatch, an ankle bracelet, a ring, and/or jewelry that includes the desired components and is configured to connect to the smartphone 10 via the local interface 45.

The external device 15 can also be configured to be part of the smartphone 10 itself For example, the sensor 60 can be disposed directly on the smartphone 10 (e.g., the sensor 60 can be integrated into the structure of the smartphone 10 itself).

The sensor 60 can be configured to collect medical information relating to the patient 20. For example, the sensor 60 can be configured to capture an ECG, capture an electroencephalogram (EEG), determine blood glucose levels, determine blood pressure, determine a heart rate, determine adrenaline levels, detect temperature at one or more discrete locations on the patient 20, record circadian rhythms, detect hemoglobin levels, collect ultrasound information, capture a photograph (e.g., visible light, and/or infrared) and/or detect breathing patterns. Other medical information can also be detected by the external device 15. The sensor 60 can include more than a single sensor (e.g., three sensors to capture an ECG), and can be attached to the patient 20 using adhesive (e.g., pressure sensitive adhesive straps), pads, wrist straps, leg straps, and/or arm bands. The sensor 60 can also be configured as a biochemical odor detector that can, for example, detect ovulation, impending seizures, impending migraine headaches, foot odor, halitosis, underarm odor, etc. The sensor 60 can also be configured to detect stress levels in the patient 20 by, for example, detecting a heart rate, a temperature, a perspiration level, and a metabolic rate of the patient 20.

The sensor 60 can also be configured to detect information other than medical information. For example, the sensor 60 can be configured to detect smoke, fire, carbon monoxide, temperature, freezing temperatures, moisture, explosive gases, light, etc. The sensor 60 can also be configured as a breathalyzer configured to determine a blood alcohol level of the patient 20.

The local interface 65 can be configured to provide data communication with the smartphone 10. Preferably, the local interface 65 is configured to communicate with one or more other devices located in the general proximity of the external device 15. The local interface 65 can be wired (e.g., USB, IEEE 1394, and proprietary) and/or wireless (e.g., IEEE 802.11, 802.15, infrared). The local interface 65 can be configured to send information to and/or receive information from the smartphone 10. In the event that the external device is connected to the smartphone 10 via a wired connection (e.g., a 30-pin IPHONE dock connector), the local interface 65 can be configured to communicate with the smartphone 10 via unused pins of the wired connection.

The external device 15 can also include a processor 70 that is configured to read and execute computer readable instructions from a computer readable medium to perform the functionality described herein. The processor 70 can be configured to control the sensor 60, receive data from the sensor 60, and/or control the local interface 65. For example, the processor 70 can be configured to operate a blood pressure cuff and to receive and process the feedback received therefrom. The processor 70 can be configured to store (e.g., in a memory) and/or process the information provided by the sensor 60 before providing it to the smartphone 10. For example, the processor 70 can be configured to amplify and filter ECG signals received from the sensor 60. The processor 70 can be omitted (e.g., raw data can be provided directly to the smartphone 10).

The external device 15 can also include a feedback device 75. The feedback device 75 can be, for example, visual (e.g., LCD and/or LED), audible (e.g., signal tones and/or verbal feedback), and/or tactile (e.g., vibration). The feedback device 75 can be configured provide feedback to the user. For example, the feedback device 75 can provide feedback to the user informing the user whether the external device 15 is positioned correctly, is actively collecting medical information, and/or is finished capturing medical information.

The external device 15 can also be configured to perform self-testing and/or calibration using a known feedback signal. For example, the external device 15 can be configured to produce a known signal that is detected by the sensor 60. Once the feedback signal is received, the received signal can be compared to the known signal to generate a transfer function that can be used to compensate for noise and/or distortion. In an embodiment of the external device 15 that is used to generate an ECG, a known electrical signal (e.g., a sine wave) can be generated by the external device 15 and detected by the sensor 60.

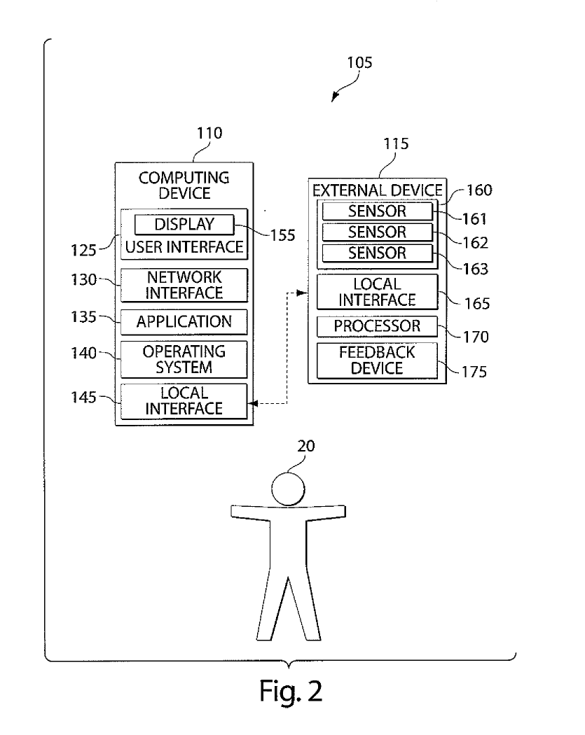

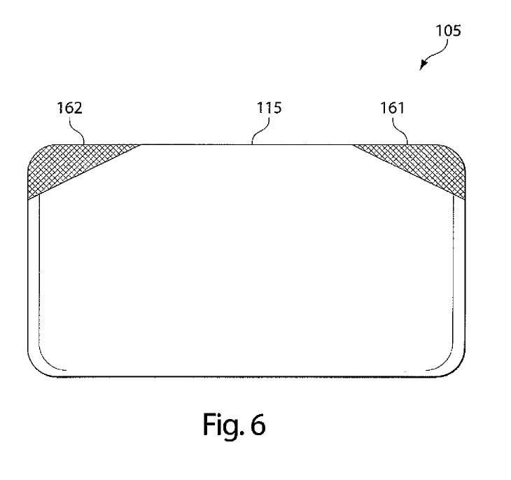

Referring to FIG. 2-10, exemplary embodiments of the system 5 are shown. A system 105 includes smartphone 110, and external device 115 and can be configured to capture an ECG of the patient 20. The remainder of the description of FIGS. 2-10 assumes that the smartphone 110 is an APPLE IPHONE, manufactured by APPLE CORPORATION of Cupertino, Calif., although the smartphone 110 can be any of the other various computing devices described herein. Furthermore, the remainder of the description of FIGS. 2-10 assumes that the external device 115 is a skin that includes test leads configured to detect electrical signals used in capturing an ECG, although the external device 115 can be any of the other various external devices described herein. Furthermore, many, if not all, of the features and variations described with respect to FIGS. 2-10 can also apply to the system 5.

The IPHONE 110 includes a user interface 125, a network interface 130, an application 135, an operating system 140, and a local interface 145. The user interface 125 preferably includes a touch-sensitive display 155. The network interface 130 can be configured as a wireless interface that is configured to connect to a GSM cellular network, and/or an 802.11 wireless network. The application 135 is an application that is configured to capture an ECG related to the patient 20. The operating system 140 can be IPHONE OS. The local interface 145 can be configured as an IPHONE 30-pin docking connector, and/or a Bluetooth interface.

The skin 115 can be configured as a vinyl and/or rubber shell that wraps the IPHONE 110. Preferably, the skin 115 is configured not to interfere with the operation of the IPHONE 110. For example, the skin 115 can include one or more cutouts that allow access to the features of the IPHONE 110 (e.g., a cutout 200 allowing access to the display 155). The skin 115 can also be configured such that the IPHONE 110 can be worn by the patient 20 (e.g., on a belt and/or harness). The skin 115 can also be configured to fit within a larger case (e.g., a holster) that is configured to attach to a belt worn by the patient 20.

The skin 115 can be configured to include a local interface 165. The local interface 165 can be configured to communicate with the local interface 145 of the IPHONE 110. The local interface 165 can be configured to communicate with the local interface 145 via, for example, a 30-pin IPHONE docking connector, a personal area network (e.g., IEEE 802.15 (e.g., Bluetooth)), and/or a wide area network (e.g., IEEE 802.11). The local interface 165 can include the necessary hardware to control operation, and/or control can be provided by another component (e.g., a processor 170). In an embodiment of the local interface 165 that uses a wired connection, the local interface 165 can be positioned to mate with the local interface 145 when the skin 115 is installed on the IPHONE 110. Alternatively, the skin 115 can also be configured not to interfere with access to the local interface 145 when the skin 115 is installed on the IPHONE 110. Furthermore, the local interface 165 can be configured to simultaneously allow a wired connection between multiple external devices and the local interface 145 simultaneously (e.g., the local interface 165 can be configured as a pass-through device).

In certain embodiments of the system 105, the local interface 145, 165 can be configured to use physical impulses to transmit information. For example, the local interface 145 can be an accelerometer included in the IPHONE 110, and the local interface can be a device configured to create modulated vibrations that represent an ECG signal. The modulated impulses created by the local interface 165 can be detected by the local interface 145 (e.g., the accelerometer), and decoded by, for example, the application 135. The local interface 145 (and/or the processor 170) can also be configured to adjust a timescale of the ECG to reduce the bandwidth used to send the ECG such that the ECG signal can be transmitted in real-time.

In other embodiments of the system 105, the local interface 145, 165 can be configured to transmit information via a headphone/microphone interface included on the IPHONE 110. The local interface 145 can be the headphone/microphone receptacle on the IPHONE 110 and the local interface 165 can be configured as a mini-plug that is configured to mate with the headphone/microphone receptacle. For example, the local interface 165 can configured as a modem that communicates analog and/or digital information with the local interface 145. In addition, in a similar manner, information can also be transmitted between the local interface 145, 165 using a tone generator (e.g., speaker) and a built-in microphone included in the IPHONE 110. For example, the built-in microphone can detect and process modulated audible signals used to transmit information (e.g., 20 Hz-100 kHz).

In yet another embodiment of the system 105, the local interface 145, 165 can be configured to transmit information using a radio frequency identification (RFID) tag. For example, each of the sensors 160 can include an RFID tag that is configured to encode and transmit information detected by the sensor 160. The RFID tags can be configured to transmit the encoded information in response to an interrogation signal provided by the local interface 145. In certain RFID embodiments, a battery may be omitted from the RFID tag.

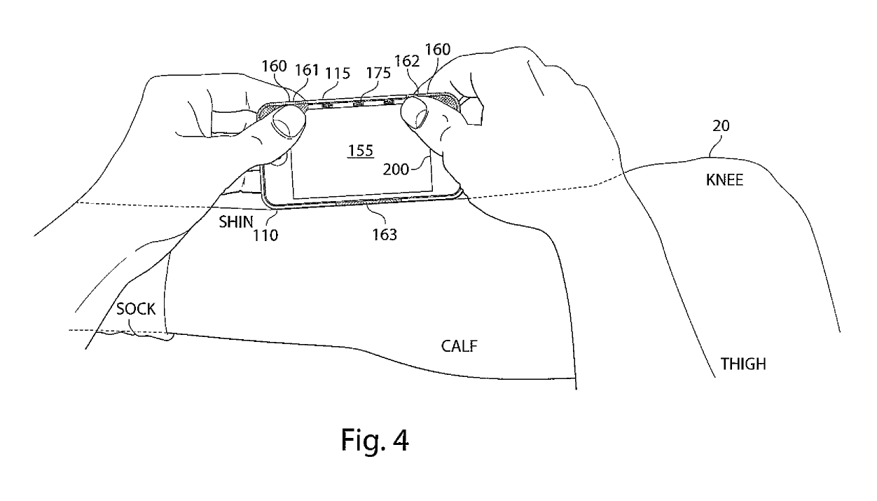

The skin 115 can be configured to include sensor 161, 162, 163. The sensor 161, 162, 163 are preferably electrodes (e.g., conductive strips) that can are configured to detect electrical signals produced by the patient 20. One or more of the sensor 161, 162, 163 can also be configured to include a file-like cross-hatched area that can rough up the skin of the patient 20 to enhance the electrical connection made between the skin and the sensor 161, 162, 163. The sensor 161, 162, 163 can be unipolar or bipolar. The sensor 161, 162 can be configured to extend to a front and/or a back of the IPHONE 110. For example, the sensor 161 and 162 may only be on the back of the IPHONE 110 such that only electrical signals from the fingers (i.e., and not the thumbs) of the patient 20 are detected when held as shown in FIG. 4. The skin 115 can also be configured to include visual indicators (e.g., indicia, finger depressions, indentations, grooves, and bumps) that can be used by the patient 20 to properly position the skin IPHONE 110.

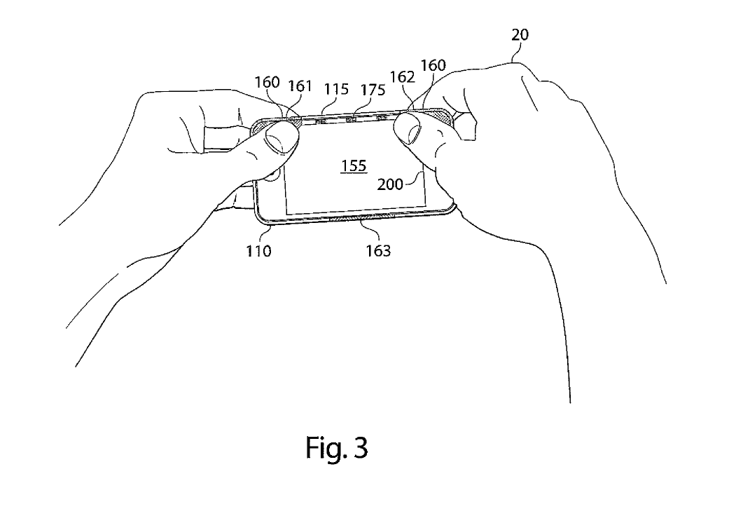

The sensor 161, 162, 163 can be configured to make physical contact with the patient 20 in several locations. For example, a left index finger can be placed on the sensor 161, a right index finger can be placed on the sensor 162, and a leg of the patient can be placed in contact with the sensor 163. An exemplary configuration of the sensors 161, 162, and 163 is shown in FIGS. 3-4, although other configurations are possible. As shown in FIGS. 3-4, the sensor 161 is positioned in a first corner (relative to the display 155) of the IPHONE 10, the sensor 162 is positioned in a second corner (relative to the display 155) of the IPHONE 10, and the sensor 163 is position on a side of the IPHONE 10 (e.g., on the “bottom” of the display 155 when the display 155 is operated in a landscape orientation).

Preferably, the location of the sensor 161, 162, 163 are such that the patient 20 can comfortably squeeze the sensor 161, 162, between the thumb and forefinger of each hand. Preferably, the sensors 161, 162 are configured such that the hands of the patient 20 do not contact one another, and such that the patient 20 can view the display 155. The sensor 163 can be configured to be pressed against the body of the patient 20. For example, the sensor 163 can be placed against the shin of the patient 20 (e.g., as shown in FIG. 4). Alternatively, the sensor 163 can be placed against other locations of the patient 20 (e.g., on the chest, arm, thigh, leg, and/or abdomen). Preferably, the sensor 161, 162, 163 are configured such that when all three are the sensors are being used, the patient is still able to view the display 155.

The skin 115 can also include a indicator 175 that functions as the feedback device 175. The indicator can consist of a series of LEDs (and/or a multi-segment LED display) that illuminate as a function of the strengths and/or quality of the signals detected by the sensor 161, 162, 163. For example, the quantity of LEDs illuminated can be a function of the strengths of the ECG signal. Other feedback methods can also be used such as audible and/or tactile feedback methods. The indicator 175 can be configured to indicate the overall intensity and/or quality of the signals detected by these sensor 161, 162, 163, and/or can be configured to indicate the intensity and/or quality of the signals detected by each individual sensor (e.g., feedback device 176, 177, 178 can relate to the sensor 161, 162, 163, respectively). The indicator 175 can also be configured to indicate which sensor(s) are not functioning properly. The indicator 175 can also inform the patient 20 once the duration of the captured ECG is sufficient (e.g., after a predetermined number of cycles and/or predetermined time).

The skin 115 can include the processor 170 that can be configured to control, for example, the indicator 175 and/or the local interface 165. The processor 170 is configured to execute computer readable instructions that cause the processor 170 to carry out the functionality described herein. The processor 170 can be configured to receive, process, and/or store signals provided by the sensor 161, 162, 163. For example, the processor 170 can be configured to receive signals from the sensor 161, 162, 163, filter the signals to remove noise, and to temporarily store the signals prior to transmission to the IPHONE 110 (e.g., in a memory). The processor 170 can also be configured to control the operation of the indicator 175 (e.g., instead of the IPHONE 110 controlling the indicator 175 directly).

In addition to the local interface 165 providing self control, the processor 170 can also be configured to control the operation of the local interface 165. For example, the processor 170 can be configured to process the information received from the processor 170 and to provide it to the IPHONE 110. This process can include the digitization of an analog signals received from the sensor 161, 162, 163 into a format recognizable by the IPHONE 110. Additionally, in a wired embodiment of the local interface 165, the processor 170 can also communicate the digitized signals to the IPHONE 110 via one or more pins of the 30-pin IPHONE docking connector. In a wireless embodiment of the local interface 165, the processor 170 can communicate the digitized signals to the IPHONE 110 via an IEEE 802.11 and/or 802.15 (e.g., Bluetooth) connection. The processor 170 can also be configured to assign one or more priorities to the information sent to the local interface 145.

The processor 170 can be configured to send information to the local interface 145 in a format recognizable by the IPHONE 110. For example, the processor 170 can be configured to send information to the local interface 145:
Via unused pins/channels in the local interface 145 (e.g., different pins are assigned to different ones of the sensor 161, 162, 163);
Via already in-use pins/channels in such a manner that the information is differentiated from other signals. Examples of differentiation can include:
   Varying frequencies to identify specific signals (e.g., different frequencies are assigned to different ones of the sensor 161, 162, 163);
   Using unique identifiers and/or meta tags to identify specific signals (e.g., different unique identifiers are assigned to different ones of the sensor 161, 162, 163);
   Time-division multiplexing can be used (e.g., different predetermined time slots can be assigned to different ones on the sensor 161, 162, 163); and

The application 135 is preferably an application that has been written for the IPHONE using an IPHONE software development kit (SDK) provided by Apple, Inc of Cupertino Calif. The application 135 can be written using Obective-C, although other programming languages can be used (e.g., C, C++, C#, Java, Pearl, PHP, Visual Basic, Python, SQL, and Pascal). The application 135 can be configured to interface with the skin 115 to generate an ECG of the patient 20.

The application 135 is configured to receive signals provided by the skin 115 (e.g., via the local interface 145), process the received signals, and display the received signals to the patient 20 (e.g., via the display 155). Preferably, the application 135 is similar to software found in ECG machines with changes made to interface with the operating system 140.

The application 135 can be configured to use the processing power of the IPHONE 110 to detect, analyze, store, display, and/or transmit historical baseline and current ECGs of the patient 20. For example, the application 135 can be configured to acquire the signals from skin 115, process the signals, and display a trace representative of the signals (e.g., display an ECG). The application 135 can also store and/or display metadata relating to captured ECGs. For example, the metadata can include the name, age, weight of the patient 20, and a time/date indicating when the ECG was captured.

The application 135 can be configured to store (e.g., in a memory) ECG information as a record that can be saved for subsequent examination and analysis. The application 135 can also be configured to transmit a captured ECG via, for example, the network interface 130 (e.g., by phone, e-mail, SMS, and text message), and/or via the local interface 145 (e.g., via a wired connection, a Bluetooth connection, and/or an 802.11 connection). The information can be transmitted to, for example, police, EMTs, a physician responsible for treating the patient 20, and/or a third-party provider that can accept and store the information generated for immediate analysis and possible immediate or future corrective action. The application 135 can also be configured to send ECG information to a printer and/or remote storage (e.g., a remote memory) using the network interface 130 and/or the local interface 145.

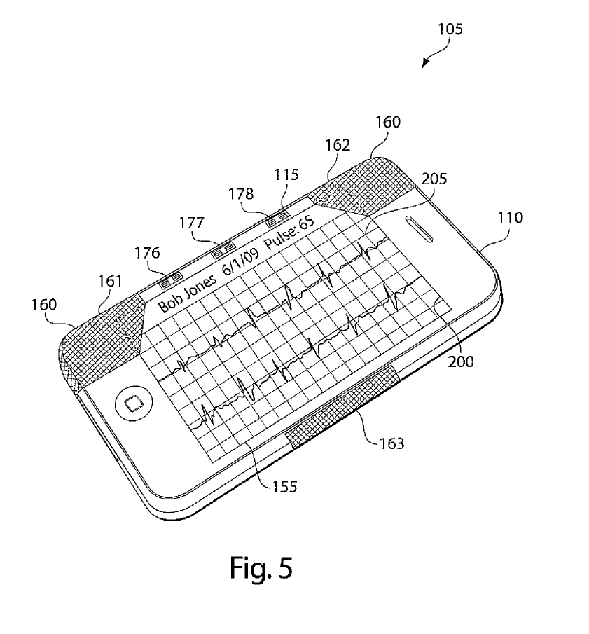

The application 135 can be configured to store (e.g., in a memory) and/or display a baseline (e.g., non-eventful) ECG of the patient 20 such that a newly captured ECG can be compared and diagnosed. In this manner, a physician can more accurately detect whether a newly captured ECG includes a medically significant event. For example, the application 135 can be configured to overlay and/or compare a newly captured ECG over a baseline ECG on the display 155. The application 135 can also be configured to allow the patient 20 to select the option of displaying and/or transmitting (e.g., via the network interface 130) the most recent baseline ECG of the patient 20 on top of (or beneath of) each current ECG. Additionally, the application 135 can also display vertical and horizontal grids as a background to enhance visual comparison and/or analysis of captured ECGs (e.g., the grid 205 in FIG. 5).

The application 135 can be configured to execute automatic diagnostic protocols that can identify abnormal ECGs and, for example, when warranted by an egregious or eventful reading, alert the patient 20, and/or simultaneously alert a third-party. For example, the application 135 can detect an abnormal ECG, dial 911 via the network interface 130, play a prerecorded message informing the 911 operator of the emergency, and provide location information to 911 (e.g., using a GPS signal provided by the IPHONE 110). Additionally, the application 135 can also call third-party call messaging services, (e.g., such as those provided by CallingPost Communications of Augusta, Ga.), that can automatically relay a prerecorded message to many people simultaneously. The alerts provided by the application 135 can be modified based on the location of the patient 20 and/or time of day or night. For example, the application 135 can also be configured to call other locations based on the specific location of the IPHONE 110 and the location of the closest emergency services as identified by GPS service and map providers.

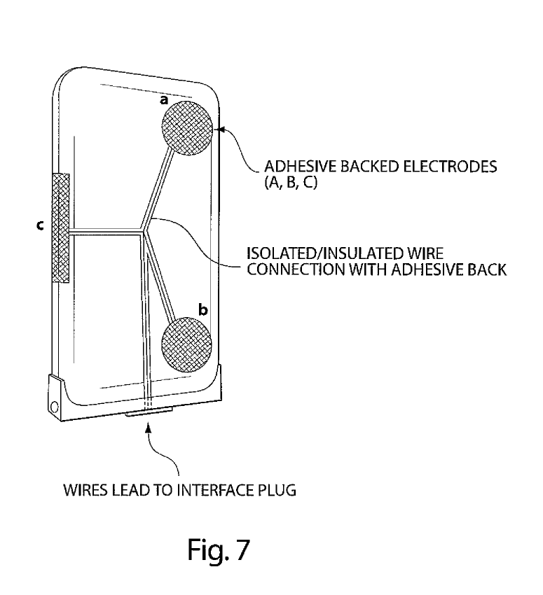

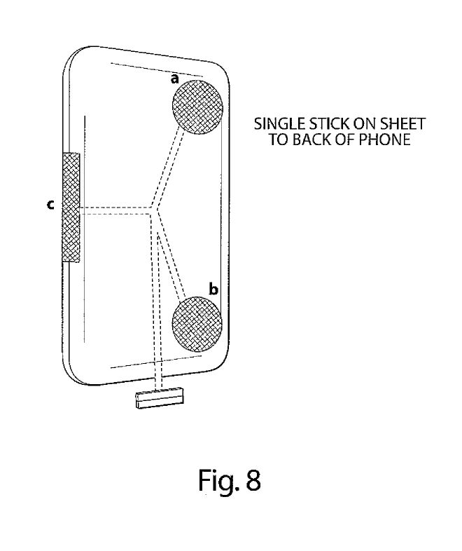

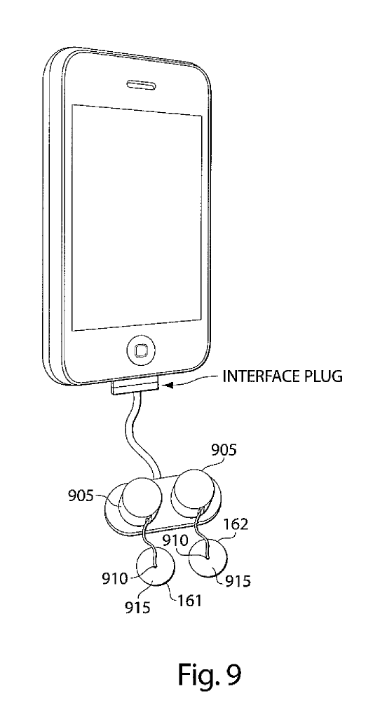

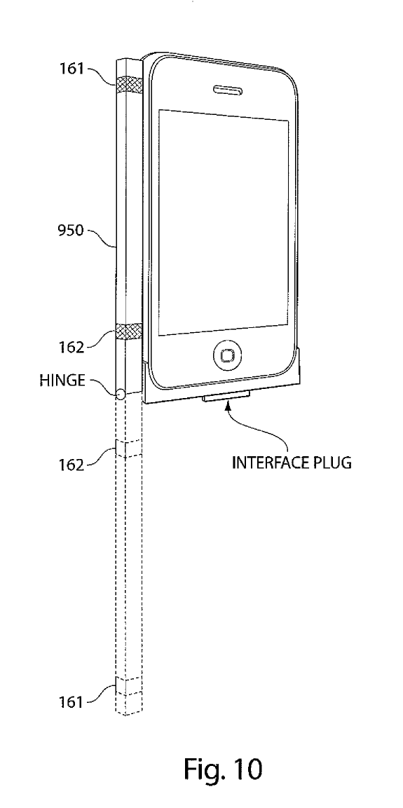

While a skin 115 has been described herein, other embodiments are possible. For example, the following variations are possible:
The sensor 161, 162, 163 can be built-in to the IPHONE 110 unit itself and/or or permanently adhered to strategically located areas on the IPHONE 110. For example, the sensor 161, 162, 163 can be snapped and/or adhered to an insulated metal or plastic web that can be used with the IPHONE 110. Preferably, each of the sensor 161, 162, 163 are electrically isolated from each other and are individually connected to a local interface that can plug into the IPHONE 110 when the web properly is snapped on to an IPHONE 110. An example of this embodiment is shown in FIG. 7.
The sensor 161, 162, 163 can be configured on a sheet of multi-layered stick-on material that is adhered to the IPHONE 110. The sheet can be configured such that the sensors 161, 162, 163 are embedded therein. Preferably, the sheet is selectively electrically insulating or conducting, such that once placed on the IPHONE 110, strategically located conductive surfaces can be individually connected to the local interface 145. An example of this embodiment is shown in FIG. 8.
The sensor 161, 162, 163 can be configured to use wires that are stored in spring-loaded, automatically-retracting wind-up spools (e.g., spools 905) that can be attached to the IPHONE 110 and/or built into the skin 110. The wires can include the sensor 161, 162, 163 directly at the end of each wire, and/or can use a receptacle to connect to a test lead. For example, the wires can include snaps (e.g., snaps 910) which are configured to connect to test leads (e.g., test leads 915). An example of an embodiment that includes two self-retracting spools is shown in FIG. 9.
Each of the sensor 161, 162, 163 can be mounted on one or more supports (e.g., support 950). Such supports can be shaped to help position each electrode properly on the body of the patient 20 relative to each other and relative to the body of the patient 20 so as to receive a clean and true ECG of the patient 20. Such supports can be shaped as i) two or more physically connected articulating, hinged and/or pivoting arms on which can be mounted one or more of the sensor 161, 162, 163, ii) a single rigid arm on which can be mounted one or more of the sensor 161, 162, 163, iii) a rigid triangle on which can be mounted one or more of the sensor 161, 162, 163; and iv) A flat folding pad on which can be mounted one or more of the sensor 161, 162, 163 that can be connected to one end of the IPHONE 110, or the skin 115, using a hinge that would be attached to one side and to one end of the IPHONE 110. An example of this embodiment is shown in FIG. 10.
The sensor 160 can include twelve separate sensors (e.g., leads). Each of the sensors can be attached to the patient 120 using adhesive, or, can be attached to a tight-fitting undershirt, harness, strap, and/or other piece of clothing. Each of the sensors can be connected to a single transmitter and/or can include a built-in transmitter that is configured to send the electrical signal from the sensor to the IPHONE 110 (e.g., using a Bluetooth signal). Each of the sensors can also include a picture illustrating where the sensor is to be positioned on the body of the patient 120. The signal transmitted from each sensor 160 can include a unique identifier such that the IPHONE 110 can determine which electrode (and thus, location) a signal is transmitted from.

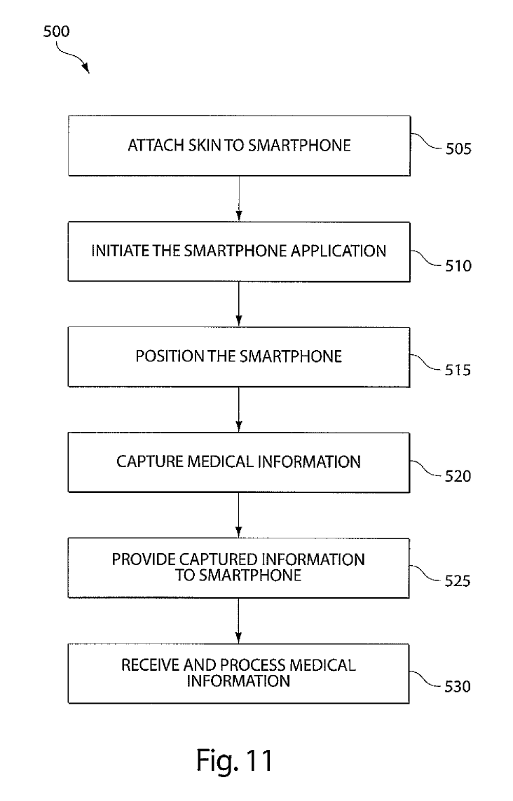

In operation, referring to FIG. 11, with further reference to FIG. 2-4, a process 500 for capturing an ECG using the system 105 includes the stages shown. The process 500, however, is exemplary only and not limiting. The process 500 may be altered, e.g., by having stages added, removed, altered, and/or rearranged. While the process 500 is described with respect to the embodiment of the system 5 shown in FIGS. 2-4, the process 500 can also be used with the system 5 shown in FIG. 1.

At stage 500, the skin 115 is attached to the IPHONE 110. Depending on the configuration of the skin 115, it can be attached using different methods. For example, if the skin 115 is made of an elastic material, it can be stretched around the IPHONE 110. Alternatively, if the skin is configured in a rigid configuration, the skin can be attached using as a “clamshell” and/or by sliding the skin 115 over the IPHONE 110.

At stage 510, the patient initiates the application 135. Preferably, the patient 20 navigates to the application 135 through one or more menus presented by the operating system 140 of the IPHONE 110. The patient 20 can initiate an ECG using menus and/or buttons presented by the application 135 on the display 155. The application 135 can also be configured to automatically launch upon detection of an ECG signal from the sensor 161, 162, 163.

At stage 515, the patient 20 positions the IPHONE 110 to capture an ECG. Preferably, the left and rights thumbs of the patient 20 are placed on the sensors 160 and 161, respectively. The patient 20 preferably places the sensor 163 in contact with another portion of the body. For example, the sensor 163 can be placed against the shin (e.g., as shown in FIG. 4), the thigh, the chest, and/or the abdomen of the patient. Preferably, the IPHONE 110 is position such that the patient is able to view the display 155 while capturing an ECG. In other embodiments of the sensor 161, 162, 163 that are not disposed on the skin 115 and use a wire to connect to the skin 115, the sensor 161, 162, 163 can be attached to the body of the patient 20 using, for example, adhesive (e.g., all of the sensor 161, 162, 163 can be placed on the chest of the patient 20). The application 135 can indicate to the patient 20 whether the IPHONE 110 is positioned correctly by providing feedback via the display 155 and/or the feedback device 175.

At stage 520, the patient 20 captures an ECG. The patient 20 preferably holds the IPHONE 110 as described in stage 510 for a specific amount of time. For example, the patient 20 can maintain the position of the IPHONE 110 as instructed by the application 135 on the display 155. The application 135 can be configured to determine the optimum time desired to collect a high-quality ECG (e.g., two complete cycles, and/or for 12 seconds). In an embodiment where the operation of the sensor 161, 162, 163 is controlled by the processor 170, the application 135 can cause the processor 170 to collect the desired signals.

During stage 520 the sensor 161, 162, 163 detect electrical signals generated by the heart of the patient 20. The sensor 161, 162, 163 forward the signals to the processor 170 which can process the signals, if desired. For example, the processor 170 can digitize the signals, filter the signals, store the signals, perform digital signal processing on the signals, de-noise the signals, etc. The processor 170 can store the collected information in a memory, and/or forward the information to the local interface 165. In an embodiment where the processor 170 is omitted, the signals detected by the sensor 161, 162, 163 can be communicated directly to the local interface 165.

During stage 525, the local interface 165 communicates the information collected by the sensor 161, 162, 163 to the local interface 145 of the IPHONE 115. The local interface 165 can communicate the collected information via a wired and/or wireless interface. In a wired embodiment, the local interface 165 can communicate the information to the local interface 145 via unused pins and/or using other methods to differentiate the information provided by the local interface 165. In a wireless embodiment, the local interface 165 can communicate the information to the local interface 145 using, for example, a personal area network (e.g., a IEEE 801.15), a wide area network (e.g., IEEE 802.11), and/or an infrared connection. The communication of the information collected by the sensor 161, 162, 163 can be performed in real-time such that the local interface 165 forwards a stream of information to the local interface 145 as it is collected by the sensor 161, 162, 163.

During stage 530, the application 135 can receive and process the ECG information from the sensor 161, 162, 163. The application 135 can display the captured ECG information on the display 155 in real-time, and or can store the captured ECG information in a memory of the IPHONE 115. The application 135 can also be configured to retrieve a baseline ECG of the patent 20, and display it concurrently with newly captured ECG information. The application 135 can be configured to align the baseline ECG and the new ECG to make a comparison easier. The application 135 can also be configured to store a newly captured ECG as the baseline. The application 135 can also be configured to detect abnormalities in a captured ECG and to provide a notification to the patient 20 and/or other parties.

The application 135 can forward ECG related information to third parties. For example, the application 135 can send a copy of a captured ECG to a third party (e.g., a medical professional) for diagnosis. The application 135 can provide a copy of a captured ECG at the direction of the patient 20, and/or in response to detecting an abnormality. The application 135 can send the captured ECG to a third party using the network interface 130 using, for example, a telephone call, an e-mail, an SMS message, a text message, etc. (e.g., via a cellular provider such as VERIZON WIRELESS).

Other embodiments are within the scope and spirit of the invention.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device or in a propagated signal), for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, (e.g., EPROM, EEPROM, and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user (e.g., the patient 20), the subject matter described herein can be implemented on a computer having a display device, e.g., a CRT (cathode ray tube) or LCD (liquid crystal display) monitor, for displaying information to the user and a keyboard (e.g., a touch screen) and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

Further, throughout this written description and claims certain components are described as being “coupled,” “connected,” and/or “in communication with” other components. These terms do not require a direct physical connection between the components. Rather, components can be “coupled,” “connected,” and/or “in communication with” other components through other non-identified components (e.g., two computers coupled to each other can include the use of a router between the two computers).

Lastly, while the description above refers to the invention, the description may include more than one invention.

What is claimed is:

1. A medical device comprising:
a housing configured to at least partially encapsulate a mobile communication device;
a sensor disposed on the housing, the sensor being configured to collect medical information relating to a patient; and
an interface coupled to the sensor and configured to communicate the collected medical information to the mobile communication device.

2. The medical device of claim 1 wherein the sensor includes a plurality of sensors configured to detect at least one of electrical activity in the patient, physiological parameters, and environmental parameters.

3. The medical device of claim 2 wherein the plurality of sensors are configured to collect information used to generate an electrocardiogram.

4. The medical device of claim 1 wherein the housing is configured as a skin that at least partially encapsulates the mobile communication device without substantially interfering with the operation of the mobile communication device.

5. The medical device of claim 1 wherein the housing includes a feedback device configured to provide to the patient feedback relating to the operation of the medical device.

6. The medical device of claim 5 wherein the feedback device is configured to provide feedback relating to at least one of a duration to collect the medical information and a quality of medical information collected.

7. The medical device of claim 1 wherein the interface is at least one of a wired interface configured to connect with an interface on the mobile communication device and a wireless interface.

8. The medical device of claim 1 further comprising a processor configured to control the operation of at least one of the sensor and the interface.

9. The medical device of claim 8 wherein the processor is configured to perform digital signal processing on information provided by the sensor.

10. The medical device of claim 1 wherein the interface comprises at least one of a radio frequency identification tag, a tone generator, and a vibration generator.

11. A tangible computer readable medium including instructions that, when executed, cause a mobile communication device to:
receive medical information relating to a patient from an external device;
perform digital signal processing on the medical information;
display on a display of the mobile communication device a visual indication that is derived from the medical information; and
store the medical information in a memory.

12. The tangible computer readable medium of claim 11 wherein the instructions are further configured to cause the mobile communication device to display an electrocardiogram relating to the patient on the display.

13. The tangible computer readable medium of claim 11 wherein the medical information is received via a local interface of the mobile communication device, wherein the local interface is at least one of a wired and a wireless interface.

14. The tangible computer readable medium of claim 11 wherein the instructions are further configured to cause the mobile communication device to determine whether abnormalities exist in the medical information.

15. The tangible computer readable medium of claim 11 wherein the instructions are further configured to cause the mobile communication device to control the operation of the external device.

16. The tangible computer readable medium of claim 11 wherein the instructions are further configured to cause the mobile communication device to provide feedback to the patient relating to at least one of the duration to collect the medical information and a quality of medical information.

17. The tangible computer readable medium of claim 11 wherein the instructions are further configured to cause the mobile communication device to transmit additional information relating to the medical information via at least one of a mobile telephone network and a data network.

18. A medical device comprising:
a medical information collection device comprising:
a first local interface;
a sensor in communication with the first local interface and configured to collect medical information relating to a patient, wherein the sensor is configured to provide the medical information to the first local interface;
a mobile communication device comprising:
a second local interface configured to communicate with the first local interface;
a first processor in communication with the second local interface and configured to receive the medical information, the first processor being configured to perform digital signal processing on the medical information;
a display in communication with the processor and being configured to display an image derived at least in part from the medical information;
a network interface in communication with the processor and configured to communicate with at least one of a mobile telephone network and a data network; and
a memory in communication with the first processor and being configured to store the medical information,
wherein the medical information collection device is configured as a housing that at least partially encapsulates the mobile communication device.

19. The medical device of claim 18 wherein the sensor comprises a plurality of sensors configured to detect at least one of electrical activity in the patient, physiological parameters, and environmental parameters.

20. The medical device of claim 19 wherein the first processor is configured to generate an electrocardiogram using the medical information.

21. The medical device of claim 18 wherein the housing is configured as a skin that at least partially encapsulates the mobile communication device without interfering with the operation of the mobile communication device.

22. The medical device of claim 18 wherein the medical device further comprises a feedback device configured to provide feedback relating to at least one of a duration to collect the medical information and a quality of medical information collected.

23. The medical device of claim 18 wherein the first and second interfaces are configured to connect with each other using at least one of a wired and a wireless interface.

24. The medical device of claim 18 wherein the medical information collection device further comprises a second processor configured to control the operation of at least one of the sensor and the first interface.

25. The medical device of claim 24 wherein the second processor is configured to perform digital signal processing on information provided by the sensor.

26. A medical device comprising:
a housing configured for use with a mobile communication device;
a sensor disposed on the housing, the sensor being configured to collect medical information relating to a patient; and
an interface coupled to the sensor and configured to communicate the collected medical information to the mobile communication device.

27. A mobile communication device comprising:
a housing;
a display;
a sensor disposed on the housing and being configured to collect medical information relating to a patient;
a processor in communication with the sensor and the display; and
a memory including computer readable instructions that are configured to, when executed by the processor, cause the processor to
receive the collected medical information;
process the collected medical information; and
display an image on the display as a function of the collected medical information.

28. A medical device for use with a mobile communication device comprising:
an adhesive skin configured adhere to at least a portion of the mobile communication device;
a sensor disposed on the adhesive skin, the sensor being configured to collect medical information relating to a patient; and
an interface coupled to the sensor and configured to communicate the collected medical information to the mobile communication device.

29. A medical device for use with a mobile communication device comprising:
a base portion configured to attach to the mobile communication device;
an arm connected to the base portion;
a sensor disposed on the arm, the sensor being configured to collect medical information relating to a patient; and
an interface coupled to the sensor and configured to communicate the collected medical information to the mobile communication device.

30. A device comprising:
a housing configured to at least partially encapsulate a mobile communication device;
a sensor disposed on the housing, the sensor being configured to collect environmental information; and
an interface coupled to the sensor and configured to communicate the collected environmental information to the mobile communication device.

31. The device of claim 30 wherein the sensor is at least one of a smoke detector, fire detector, carbon monoxide detector, a thermometer, a moisture detector, and a photocell.
